(19) United States
(12) Patent Application Publication
(10) Pub. No.: US 2019/0201474 A1
(43) Pub. Date: Jul. 4, 2019

(54) HERBAL OIL FORMULATION FOR TOPICAL USE AND MEDICINAL APPLICATIONS THEREOF

(71) Applicants: Dinesh Upendra NAYAK, Thane, Maharashtra (IN); Ravindranath AITHAL, Dakshin Kannada, Karnataka (IN)

(72) Inventors: Dinesh Upendra NAYAK, Thane, Maharashtra (IN); Ravindranath AITHAL, Dakshin Kannada, Karnataka (IN)

(21) Appl. No.: 16/326,776

(22) PCT Filed: Oct. 6, 2017
(86) PCT No.: PCT/IB2017/056170

§ 371 Date: Feb. 20, 2019

(30) Foreign Application Priority Data
Oct. 14, 2016 (IN) 201621035264

(51) Int. Cl. A61K 36/9064; A61K 36/63; A61K 36/61; A61K 36/484; A61K 36/71; A61K 36/23; A61K 9/00; A61P 17/02; A61P 31/10

(52) U.S. Cl. CPC A61K 36/9064; A61K 36/63; A61K 36/61; A61K 36/484; A61P 31/10; A61K 36/23; A61K 9/0014; A61P 17/02; A61K 36/71

(57) ABSTRACT

The present disclosure relates to a herbal oil formulation based on bark extract of Heterophragma roxburghii, that can be applied topically and can be used to for treating and curing various skin conditions and diseases, all types of wounds, and other therapeutic conditions associated with diminished blood flow in humans and animals. The disclosed topical herbal oil formulation provides an inexpensive alternative healing therapy for various therapeutic conditions such as, but not limited to, diabetic gangrene, dry gangrene, wet gangrene, athlete's foot, burn wounds, diabetic foot ulcer, bed sore, chronic open wounds, wounds caused by snake bite, and gangrene formed due to cellulitis.

### TECHNICAL FIELD

The present disclosure pertains to herbal oil formulation for topical application. In particular, the present disclosure pertains to topical herbal oil formulation based on bark extract of Heterophragma roxburghii, capable of treating various skin conditions and diseases, all types of wounds, and other therapeutic conditions associated with diminished blood flow in humans and animals.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Diabetic gangrene is characterized by obstructed supply of blood due to tissue damage or death of body tissue. It is caused due to an underlying injury, infection or illness resulted from poor blood supply to body parts of diabetic patients. Diabetic gangrene usually affects the extremities like fingers, toes and limb. About 1 in 3 diabetic individuals are more likely to develop a foot ulcer in their lifetime, which if left untreated may develop into diabetic foot gangrene. About 85% of all limb amputations in diabetic individuals are followed by foot ulcers. Every 30 seconds, a lower limb amputation is carried out due to diabetes throughout the world. The mortality rate due to diabetic foot gangrene is just next to cancer death rate.

Foot ulcer is an open sore on the foot, and it can be a shallow red crater that involves only the surface skin. Foot ulcer can also be very deep. A deep foot ulcer may be a crater that extends through the full thickness of the skin. It may involve tendons, bones and other deep structures. People with diabetes and people with poor blood circulation are more likely to develop foot ulcers. In people with these conditions, even a small foot ulcer can become infected if it does not heal quickly. If an infection occurs in an ulcer and is not treated right away, it can develop into an abscess (a pocket of pus, a spreading infection of the skin and underlying fat (cellulitis), a bone infection (osteomyelitis), or gangrene.

Gangrene is a condition that occurs when body tissue dies. It is caused by loss of blood supply due to an underlying illness, injury, and/or infection. Fingers, toes, and limbs are most often affected by gangrene. Gangrene can also occur inside the body, damaging organs and muscles. There are different types of gangrene and all require immediate medical attention.

Dry gangrene is more common in people with diabetes and autoimmune diseases, which usually affects hands and feet of human body. It develops when blood flow to the affected area is impaired, usually as a result of poor circulation. In this type, the tissue dries up and may be brown to purplish-blue to black in color and often falls off. Unlike other types of gangrene, infection is typically not present in dry gangrene. However, dry gangrene can lead to wet gangrene if it becomes infected.

Unlike dry gangrene, wet gangrene almost always involves an infection. Injury from burns or trauma where a body part is crushed or squeezed can rapidly cut off blood supply to the affected area, causing tissue death and increased risk of infection. The tissue swells and blisters and is called “wet” because of pus. Infection from wet gangrene can spread quickly throughout the body, making wet gangrene a very serious and potentially life-threatening condition if not treated quickly.

A chronic open wound is one which does not heal in an orderly set of stages and in a predictable amount of time like most wounds do. Wounds that do not heal within three months are considered chronic.

Athlete's foot, known medically as tineapedis, is a common skin infection of the feet caused by fungus. It is usually a scaly, red, itchy eruption occasionally maybe weepy and oozing.

There are various types of treatments available in the field of medicine to treat gangrene and other therapeutic conditions mentioned above. For example, gangrene can be treated by removing the affected tissue, preventing infection or treating any existing infection, and treating the problem that led to gangrene developing. If gangrene is caused by a poor blood supply, surgery may be used to repair damaged blood vessels. If gangrene is caused by an infection, strong antibiotics as well as surgery may be used. However, healing and management of the conditions mentioned above pose serious challenges, and the existing therapies of modern science have various limitations and complications when it comes to treatment and cure of the aforementioned conditions.

There is thus a need in the art for a medicament, more specifically a herbal medicament that can be applied topically and used for treatment of therapeutic conditions such as diabetic gangrene, dry and wet gangrene, chronic open wounds, general wounds and injuries, wounds caused by snake bite, athlete's foot, and other therapeutic conditions associated with diminished blood flow in humans and animals.

The present invention satisfies the existing needs, as well as others, and generally overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

### OBJECTS OF THE INVENTION

It is an object of the present disclosure to provide a topical formulation based on all-natural herbal extracts useful for treating various skin conditions and diseases, all types of wounds, and other therapeutic conditions associated with diminished blood flow in humans and animals

It is a further object of the present disclosure to provide a herbal formulation based on bark extract of Heterophragma roxburghii which can be applied topically for treatment of various skin conditions and diseases, all types of wounds, and other therapeutic conditions associated with diminished blood flow in humans and animals.

It is another object of the present disclosure to provide a topical herbal formulation that provides an inexpensive alternative healing therapy for various skin conditions and diseases, all types of wounds, and other therapeutic conditions associated with diminished blood flow, which does not have undesirable side-effects.

It is another object of the present disclosure to provide a topical herbal formulation that can be used for treating various therapeutic conditions and diseases such as, diabetic gangrene, dry gangrene, wet gangrene, athlete's foot, burn wounds, diabetic foot ulcer, bed sore, chronic open wounds, wounds caused by snake bite and gangrene formed due to cellulitis.

It is another object of the present disclosure to provide a process for preparation of a topical herbal formulation useful for treating various skin conditions and diseases, all types of wounds, and other therapeutic conditions associated with diminished blood flow in humans and animals.

It is yet another object of the present disclosure to provide a method of treating various skin conditions and diseases, all types of wounds, and other therapeutic conditions associated with diminished blood flow in humans and animals using a topical herbal formulation.

### SUMMARY OF THE INVENTION

Aspects of the present disclosure relate to topical herbal oil formulation capable of treating and curing various skin conditions and diseases, all types of wounds, and other therapeutic conditions associated with diminished blood flow in humans and animals.

In an aspect, the present disclosure provides a topical herbal oil formulation that can be used for treating various therapeutic conditions including, but not limited to, all types of gangrene, athlete's foot, skin burns, diabetic foot ulcers, bed sores, and all types of wounds, wherein the topical herbal formulation can include an oil extracted from bark of Heterophragma roxburghii.

In an embodiment, the disclosed topical herbal oil formulation, in addition to an oil extracted from bark of Heterophragma roxburghii, can further include at least one essential oil extracted from at least one plant material selected from the group consisting of Azadirachta indica, Jasminum grandiflorum, Elattaria cardamomum, Syzygium aromaticum, Glycyrrhiza glabra, Nigella sativa and Cuminum cyminum.

According to embodiments of the present disclosure, the disclosed topical herbal oil formulation can be used for treating various therapeutic conditions such as, but not limited to, diabetic gangrene, dry gangrene, wet gangrene, athlete's foot, burn wounds, diabetic foot ulcer, bed sore, chronic open wounds, wounds caused by snake bite, and gangrene formed due to cellulitis.

In another aspect, the present disclosure provides a process for preparation of a topical herbal formulation useful for treating various skin conditions and diseases, all types of wounds, and other therapeutic conditions associated with diminished blood flow in humans and animals, wherein the process can include the steps of:
(a) boiling a coarse powder of Heterophragma roxburghii bark in water to obtain an aqueous extract of Heterophragma roxburghii bark;
(b) mixing the aqueous extract of bark of Heterophragma roxburghii, Azadirachta indica oil and an aqueous extract of Jasminum grandiflorum leaves to provide a first mixture;
(c) heating the first mixture at a temperature for a period of time sufficient to obtain a concentrate;
(d) adding to the concentrate obtained in step (c) Elattaria cardamomum, Syzygium aromaticum, Glycyrrhiza glabra, Nigella sativa and Cuminum cyminum to obtain a second mixture;
(e) heating the second mixture at a temperature for a period of time to evaporate water from the second mixture so as to obtain an oil mix;
(f) filtering the oil mix obtained in step (e) to provide the topical formulation.

According to embodiments of the present disclosure, the disclosed topical herbal formulation can be formulated in various topical dosage forms including, but not limited to, gel, cream, lotions, emulsion, ointment, liquid dosage forms, etc. for external application to skin.

In another aspect, the present disclosure provides a method for treating a therapeutic condition selected from the group consisting of gangrene, athlete's foot, skin burn, diabetic foot ulcer, bed sore, and wounds in a patient suffering therefrom, wherein the method can include topical application to the patient of a herbal formulation including an oil extracted from bark of Heterophragma roxburghii.

### DETAILED DESCRIPTION OF THE INVENTION

The following is a detailed description of embodiments of the present invention. The embodiments are in such detail as to clearly communicate the invention. However, the amount of detail offered is not intended to limit the anticipated variations of embodiments; on the contrary, the intention is to cover all modifications, equivalents, and alternatives falling within the spirit and scope of the present invention as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The present disclosure relates to a herbal oil formulation based on bark extract of Heterophragma roxburghii, that can be applied topically and can be used for treating and curing various skin conditions and diseases, all types of wounds, and other therapeutic conditions associated with diminished blood flow in humans and animals. The disclosed topical herbal oil formulation can provide an inexpensive alternative healing therapy for various therapeutic conditions such as, but not limited to, diabetic gangrene, dry gangrene, wet gangrene, athlete's foot, burn wounds, diabetic foot ulcer, bed sore, chronic open wounds, wounds caused by snake bite and gangrene formed due to cellulitis.

In an aspect, the present disclosure provides a topical herbal oil formulation that can include an oil extracted from bark of Heterophragma roxburghii.

In a preferred embodiment, the disclosed topical herbal oil formulation, in addition to an oil extracted from bark of Heterophragma roxburghii, can further include at least one essential oil extracted from at least one plant material selected from the group consisting of Azadirachta indica, Jasminum grandiflorum, Elattaria cardamomum, Syzygium aromaticum, Glycyrrhiza glabra, Nigella sativa and Cuminum cyminum.

In a more preferred embodiment, the disclosed herbal oil formulation can include a blend of essential oils extracted from bark of Heterophragma roxburghii, Azadirachta indica, leaves of Jasminum grandiflorum, Elattaria cardamomum, Syzygium aromaticum, Glycyrrhiza glabra, Nigella sativa, and Cuminum cyminum.

The disclosed topical herbal formulation can exhibit anti-inflammatory, anti-biotic, anti-fungal and anti-pruritic properties, and can be used for treating and curing various therapeutic conditions such as, but not limited to, diabetic gangrene, dry gangrene, wet gangrene, athlete's foot, burn wounds, diabetic foot ulcer, bed sore, chronic open wounds, wounds caused by snake bite (e.g. Russell's viper and Cobra) and gangrene formed due to cellulitis. The disclosed topical herbal oil formulation can heal wounds through formation of granulation tissues and tissue re-epithelialization. Further, chronic non healing wounds like diabetic foot ulcer can be completely cured using the herbal oil formulation, thus amputation can be prevented.

In another aspect, the present disclosure provides a process for preparation of a topical herbal formulation that includes a blend of essential oils extracted from Heterophragma roxburghii bark, Azadirachta indica, leaves of Jasminum grandiflorum, Elattaria cardamomum, Syzygium aromaticum, Glycyrrhiza glabra, Nigella sativa, and Cuminum cyminum, wherein the process can include the steps of:
(a) boiling a coarse powder of Heterophragma roxburghii bark in water to obtain an aqueous extract of Heterophragma roxburghii bark;
(b) mixing the aqueous extract of bark of Heterophragma roxburghii, Azadirachta indica oil and an aqueous extract of Jasminum grandiflorum leaves to provide a first mixture;
(c) heating the first mixture at a temperature for a period of time sufficient to obtain a concentrate;
(d) adding to the concentrate obtained in step (c) Elattaria cardamomum, Syzygium aromaticum, Glycyrrhiza glabra, Nigella sativa and Cuminum cyminum to obtain a second mixture;
(e) heating the second mixture at a temperature for a period of time to evaporate water from the second mixture so as to obtain an oil mix;
(f) filtering the oil mix obtained in step (e) to provide the topical formulation.

In one embodiment of the process disclosed herein, an aqueous extract of bark of Heterophragma roxburghii, Azadirachta indica oil and aqueous extract of Jasminum grandiflorum leaves can be mixed in a ratio of 1:1:1 to provide the first mixture in step (b).

In another embodiment of the process disclosed herein, each of the Elattaria cardamomum, Syzygium aromaticum, Glycyrrhiza glabra, Nigella sativa and Cuminum cyminum can be added to the concentrate as obtained in step (c) in a quantity ranging from 0.1 g to 10 g per 100 ml of the concentrate.

In another embodiment, the first mixture as obtained in step (b) of the disclosed process can be heated at a temperature ranging from 95° C. to 110° C. to obtain a concentrate.

In another embodiment, the second mixture as obtained in step (d) can be heated at a temperature ranging from 100° C. to 110° C. to obtain an oil mix, which in turn can be filtered to produce the topical herbal formulation.

According to embodiments of the present disclosure, the disclosed topical herbal formulation can be formulated in various topical dosage forms including, but not limited to, gel, cream, lotions, emulsion, ointment, liquid dosage forms, etc. for external application to skin.

In another aspect, the present disclosure provides a method for treating a therapeutic condition selected from the group consisting of gangrene, athlete's foot, skin burn, diabetic foot ulcer, bed sore, and wounds in a patient suffering therefrom, wherein the method can include topical application to the patient of a herbal formulation including an oil extracted from bark of Heterophragma roxburghii.

In a preferred embodiment, the disclosed topical herbal formulation can be effectively used for treating various types of gangrene such as diabetic gangrene, dry gangrene, and wet gangrene. In case of gangrene where necrotization has occurred, the treatment can start with debridement of necrotised tissues. After debridement of the necrotised tissue, the affected area may preferably be cleaned with Betadine solution. Followed by cleaning step the so cleaned area may be left free and open to allow for excess moisture to dry away. After the cleaned affected area is dried out, the herbal oil formulation of the present disclosure can be applied onto the affected area twice daily and wounds can be kept open as no dressing is required. If one feels like dressing is necessary, it can be done with a single layer of gauze as air circulation is mandatory for the treatment to be effective.

In case of treatment of chronic open wounds and normal wounds, the affected area may be cleaned first and the herbal oil formulation can be applied onto the affected area twice daily.

For the treatment to be effective, a patient undergoing treatment according to the present disclosure may be required to alter or modify his lifestyle and diet which includes avoiding of smoking, drinking of alcohol, consumption of non vegetarian food, and certain vegetables like brinjal may also be avoided and fluid intake may be increased.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### Example

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

Preparation of Topical Herbal Oil Formulation of the Present Disclosure:

A topical oil formulation was made using the ingredients and quantities specified in below TABLE-1:

TABLE-1

| | | | | Quantity in 1 | |
|---|---|---|---|---|---|
| Botanical name | Common name | | Geographical origin | litre of oil | |
| of the Herbs | of the herbs | Part used | of the herbs used | (formulation) | |
| Heterophragma | Waras | Bark | Maharashtra, India | 1 | kg |
| roxburghii | | | | | |
| Azadirachta indica | Neem (oil) | Fruits and seeds | Gujarat, India | 1 | litre |
| Jasminum | Jasmine leaves | Leaves (freshly | Tamil Nadu, India | 1 | litre |
| grandiflorum | | crushed juice) | | | |
| Elettaria | Cardamom | Seeds | Kerala, India | 20 | g |
| cardamomum | | | | | |
| Syzygium | Clove | Flower buds | Kerala, India | 20 | g |
| aromaticum | | | | | |
| Glycyrrhiza glabra | Licorice | Root | Punjab, India | 20 | g |
| Nigella sativa | Black cumin | Seeds | Northern India | 20 | g |
| Cuminum cyminum | Cumin | Seeds | Mediterranean and | 20 | g |
| | | | eastern India | | |

Clean, dry and powder bark of Heterophragma roxburghii. Take 1 kg of coarse powder of Heterophragma roxburghii bark and mix it with 4 litres of distilled water and boil it. When the volume of the above solution reaches to one-fourth (i.e. 1 litre) of the original volume, stop boiling and filter the solution with a muslin cloth so as to obtain an aqueous extract of Heterophragma roxburghii bark. To this aqueous extract add 1 litre of neem oil and 1 litre of jasmine leaves aqueous extract and heat the so obtained mixture (total 3 litres) on low flame at 95-100 deg Celsius until the volume of the solution drops down to 2 litres to form a concentrate. Then add 20 gms each of Elattaria cardamomum, Syzygium aromaticum, Glycyrrhiza glabra, Nigella sativa, Cuminum cyminumin coarse powder form to the above obtained solution (i.e. concentrate) and continue heating on low flame at 100-110 deg Celsius till all the water has evaporated so as to obtain an oil formulation. After heating till a desired texture and aroma is attained, filter the oil, cool it to room temperature at 20-25 deg Celsius and then store the oil formulation in amber coloured containers.

### Advantages of the Present Invention

The present disclosure provides a topical herbal oil formulation based on all-natural herbal extracts useful for treating various skin conditions and diseases, all types of wounds, and other therapeutic conditions associated with diminished blood flow in humans and animals.

The present disclosure provides a herbal formulation that can be applied topically for treating various therapeutic conditions and diseases including, but not limited to, diabetic gangrene, dry gangrene, wet gangrene, athlete's foot, burn wounds, diabetic foot ulcer, bed sore, chronic open wounds, wounds caused by snake bite (e.g. Russell's viper and Cobra), and gangrene formed due to cellulitis.

The present disclosure provides a topical herbal formulation based on an oil extracted from Heterophragma roxburghii bark, which is easy to manufacture.

The present disclosure provides a topical herbal formulation based on an oil extracted from Heterophragma roxburghii bark along other herbal extracts, which formulation is completely natural, highly effective and inexpensive.

The present disclosure provides a topical herbal formulation that provides an inexpensive alternative healing therapy for various therapeutic conditions and diseases, which does not have undesirable side-effects.

The present disclosure provides a topical herbal formulation by which the more difficult to heal diabetic wounds can be completely cured as the oil formulation has broad spectrum antibiotic activity.

The present disclosure provides a topical herbal formulation that can exhibit anti-inflammatory, anti-biotic, anti-fungal and anti-pruritic properties.

The present disclosure provides a topical herbal oil formulation which can be used along with other day to day medications such as anti-diabetic, anti-hypertensives, etc without causing drug interaction problems.

The present disclosure provides a process for preparation of a topical herbal formulation useful for treating various skin conditions and diseases, all types of wounds, and other therapeutic conditions associated with diminished blood flow in humans and animals, which process is simple, cost effective and highly efficient.

1. A topical formulation comprising an oil extracted from bark of Heterophragma roxburghii.

2. The topical formulation as claimed in claim 1, further comprising at least one essential oil extracted from at least one plant material selected from the group consisting of Azadirachta indica, Jasminum grandiflorum, Elattaria cardamomum, Syzygium aromaticum, Glycyrrhiza glabra, Nigella sativa and Cuminum cyminum.

3. A topical formulation, comprising a blend of essential oils extracted from bark of Heterophragma roxburghii, Azadirachta indica, leaves of Jasminum grandiflorum, Elattaria cardamomum, Syzygium aromaticum, Glycyrrhiza glabra, Nigella sativa, and Cuminum cyminum.

4. A process for preparation of a topical formulation as claimed in claim 3, comprising the steps of:
(a) boiling a coarse powder of Heterophragma roxburghii bark in water to obtain an aqueous extract of Heterophragma roxburghii bark;
(b) mixing the aqueous extract of bark of Heterophragma roxburghii, Azadirachta indica oil and an aqueous extract of Jasminum grandiflorum leaves to provide a first mixture;
(c) heating the first mixture at a temperature for a period of time sufficient to obtain a concentrate;
(d) adding to the concentrate obtained in step (c) Elattaria cardamomum, Syzygium aromaticum, Glycyrrhiza glabra, Nigella sativa and Cuminum cyminum to obtain a second mixture;
(e) heating the second mixture at a temperature for a period of time to evaporate water from the second mixture so as to obtain an oil mix;
(f) filtering the oil mix obtain in step (e) to provide the topical formulation.

5. The process as claimed in claim 4, wherein the aqueous extract of bark of Heterophragma roxburghii, Azadirachta indica oil and aqueous extract of Jasminum grandiflorum leaves are mixed in step (b) in a ratio of 1:1:1.

6. The process as claimed in claim 4, wherein each of the Elattaria cardamomum, Syzygium aromaticum, Glycyrrhiza glabra, Nigella sativa and Cuminum cyminum is added to the concentrate in step (c) in a quantity ranging from 0.1 g to 10 g per 100 ml of the concentrate.

7. The process as claimed in claim 4, wherein the first mixture is heated at a temperature ranging from 95° C. to 110° C. to obtain the concentrate in step (c).

8. The process as claimed in claim 4, wherein the second mixture is heated at a temperature ranging from 100° C. to 110° C. to obtain the oil mix in step (e).

9. The topical formulation as claimed in claim 1, wherein the topical formulation is in a form of a gel, a cream, a lotion, an emulsion, an ointment or a liquid.

10. A method for treating a therapeutic condition selected from the group consisting of gangrene, athlete's foot, skin burn, diabetic foot ulcer, bed sore, and wounds in a patient suffering therefrom, the method comprising topical application to the patient of a herbal formulation comprising an oil extracted from bark of Heterophragma roxburghii.

11. The method as claimed in claim 9, wherein the therapeutic condition is selected from diabetic gangrene, dry gangrene, and wet gangrene.
